(19) United States
(12) Patent Application Publication
(10) Pub. No.: US 2010/0196298 A1
(43) Pub. Date: Aug. 5, 2010

(54) Coloring Materials And Their Use In Compositions, In Particular Cosmetic Compositions

(75) Inventors: Patrice ANDRE, Neuville Aux Bois (FR); Jean-Francois TRANCHANT, Marigny Les Usages (FR); Michel GARCIA, Lauris (FR)

Correspondence Address: WOODCOCK WASHBURN LLP, CIRA CENTRE, 12TH FLOOR, 2929 ARCH STREET, PHILADELPHIA, PA, 19104-2891, US

(73) Assignee: LVMH RECHERCHE, Saint Jean De Braye (FR)

(21) Appl. No.: 12/648,716

(22) Filed: Dec. 29, 2009

(30) Foreign Application Priority Data
Dec. 31, 2008 (FR) 0859168

(51) Int. Cl. A61K 8/18; A61Q 1/02

(52) U.S. Cl. 424/63

(57) ABSTRACT

The invention relates to a coloring material comprising a colored product bound to a solid substrate in which said colored product is a colored derivative of geniposide and/or genipin and/or geniposidic acid and/or an extract of a plant comprising at least one of the abovementioned products and said solid substrate is based on silicate and includes at least one metal cation. It also relates to a process for preparing this coloring material. It also relates to the use of this coloring material in the preparation of a coloring composition, in particular of a cosmetic composition, and to a method for making up the skin or superficial body growths.

### CROSS REFERENCE TO RELATED APPLICATIONS

This application claims the benefit of French Patent Application No. 0859168, filed Dec. 31, 2008, the entirety of which is incorporated herein.

### TECHNICAL FIELD

The subject matter of the present invention is directed to novel coloring materials and their use in compositions, in particular cosmetic compositions.

### BACKGROUND

In the cosmetics field, use is very often made of coloring materials of natural or synthetic origin and of inorganic or organic chemical nature. They are used mainly to produce a coloring effect on the skin or superficial body growths, as is the case for makeup products. These coloring materials can in particular be of plant origin.

Plant dyes are transparent in a liquid medium. They can be used to simply color a composition, without any coloring effect on the skin or superficial body growths. However, if it is desired to use them in makeup products or more particularly to color a surface, in order for the rendering of the colors on the skin or superficial body growths or the surface to be satisfactory, it is necessary to render them opaque. For this, they are rendered insoluble. The main technique for rendering plant dyes insoluble consists in fixing them to a solid substrate. Lakes, which are composed of three elements: a dye, a substrate, such as, for example, alumina, and a cation acting as precipitating agent, are known in particular.

Mention will be made, among the well known dyes of plant origin which can be used in cosmetics, generally after rendering insoluble, of anthocyanins, carotenoids, curcumin and chlorophylls.

It is known that a number of plants of the botanical family of the Rubiaceae are used as source for the manufacture of blue or blue-black dyes.

Mention is made in particular, among these plants, of the following Genera and species:
Genipa, in particular Genipa americana,
Gardenia, in particular Gardenia jasminoides, Gardenia ternifolia, Gardenia lucida and Gardenia erubescens,
Rothmannia, in particular Rothmannia capensis, Rothmannia longiflora and Rothmannia withfieldii,
Adenorandia, in particular Adenorandia kalbreyeri and
Cremaspora, in particular Cremaspora triflora.

The following elements should be noted for these plants:

Adenorandia kalbreyeri—The plant is distinctly equatorial (Nigeria, Congo, Angola). In the countries where the plant is present, the blue liquid extracted from the fruit acts as blue-black cosmetic product and as dye for tattoos. This is the only use known for it.

Cremaspora triflora—The plant is fairly widespread in West Africa. The green and ripe fruits are known in various parts of Africa for providing a blue-black dye used as body dye and, in the Congo, as textile dye. The ripe fruit is regarded as edible.

Rothmannia longiflora—The plant is known throughout the subregion of West Africa; it is used for blue-black tattooing (juice of the fruits) but also for dyeing, where it can replace indigo. The seeds and the flowers are also mentioned as source of black dye.

Rothmannia withfieldii—The plant is present in Mali and throughout the subregion of West Africa. The juice of the fruits and seeds is used for body paintings and for the blue-black dyeing of fabrics. The plant is mentioned in combination with Piliostigma thonningii, a plant furthermore mentioned for its use during blue colorings. The Rothmannia species are also mentioned in combination with the Gardenia species.

In Burkina-Faso, two plants are mentioned as the source of a blue dye. They are Gardenia erubescens: use of the seeds for a black dye used in cosmetics, and Gardenia ternifolia: use of the fruits in cosmetics. It should be noted that the traditional uses for body painting have in common the result of at least semipermanent colorings of the skin.

The studies carried out on the extract of the fruit of the plant Genipa americana have made it possible to isolate and characterize genipin, a colorless compound which colors on contact with the skin (Djerassi et al., J. Org. Chem., 1960, 25, 2174, and J. Org. Chem., 1960, 26, 1192).

In addition to genipin, other glycosides of the iridoid group have been isolated from the fruit of the plant Genipa americana, including in particular geniposide and geniposidic acid (Ono et al., Chem. Pharm. Bull., 2005, 53 (10), 1342-1344).

Geniposide, extracted from the pulp and seeds of the fruit of the plant Genipa americana by steeping or macerating in water, forms genipin by hydrolysis, for example by an enzyme β-glucosidase.

The blue coloring is the result of the reaction of genipin with amino acids having a primary amine group or proteins having such a group, in the presence of oxygen (FIG. 1, from Lee et al., Anal. Chem. Acta, 2003, 480, 267-274).

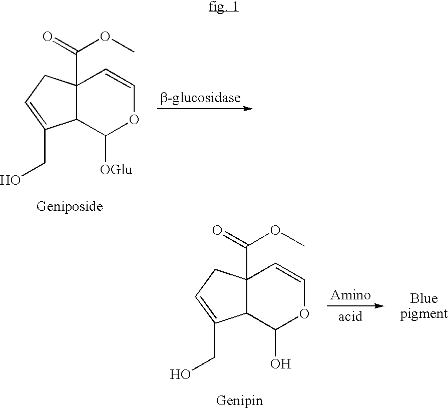

The text of the patent application JP 52-053934 discloses a coloring material obtained by reaction of genipin or one of its analogs with a compound comprising a primary amine group. The exact mechanism has not been completely elucidated.

### SUMMARY

Coloring materials comprising a colored product bound to a solid substrate, wherein said colored product is a colored derivative of a product or a mixture of products selected from the group consisting of geniposide, genipin, geniposidic acid and an extract of a plant comprising at least one of the abovementioned products, and wherein said solid substrate is based on silicate and includes at least one metal cation. Methods of making the materials are also described.

### DETAILED DESCRIPTION OF ILLUSTRATIVE EMBODIMENTS

The invention applies very particularly to the preparation of coloring cosmetic compositions, in particular intended for making up the skin or superficial body growths. It also applies to any type of coloring composition for other industrial fields, such as the field of foodstuffs, that of medicaments, inks, dyes, paints and products which can be applied in the field of graphic arts and of decoration in general.

In the context of the present invention, the expression “coloring material” denotes a substance which acts as dye in the medium into which it has been introduced or on the surface of the material or support to which it has been applied.

However, identified a number of intermediates have been identified and have put forward based on the hypothesis that the coloring material obtained is formed of water-soluble polymers having high molecular weights (Touyama et al., Chem. Pharm. Bull., 1994, 42(3), 668-673; Chem. Pharm. Bull., 1994, 42(8), 1571-1578).

It has heretofore been discovered that it is possible to obtain, starting from genipin, blues of different intensities according to the nature of the amino acid reacting with the genipin (Fujikawa, J. Ferment. Technol., 1987, 64(4), 419-24).

The coloring obtained from an extract comprising geniposide and/or genipin thus depends on the composition with regard to amino acids or with regard to proteins which are capable of reacting and which are present in the extract, the reaction medium or the support to which the extract is applied.

A red color, starting from geniposidic acid of formula (A) below, has also been obtained

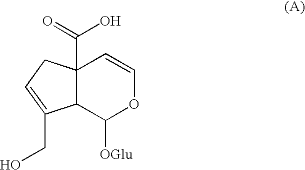

which compound is found in the same plants as those which comprise genipin and/or geniposide (Moritome et al., J. Sci. Food Agric., 1999, 79, 810-814). Geniposidic acid is, for example, extracted from the fruit of Gardenia jasminoides or is obtained by hydrolysis of the ester functional group of its precursor, geniposide. The red coloring is a result of the reaction, in an acidic medium and in the absence of oxygen, of geniposidic acid with compounds carrying a primary amine group (Moritome et al., J. Sci. Food Agric., 2002, 39(4), 345-352).

Mention will very particularly be made, among the abovementioned plants comprising genipin, geniposidic acid or their precursor of geniposide type, of Genipa americana.

Specifically, the plant Genipa americana, of the family of the Rubiaceae, is a tropical plant, the fruit of which is traditionally used by Amazonian peoples as food but which is also used externally for its beneficial effects against some types of skin irritation (in particular antipruriginous effect). The juice of this fruit is also used, under certain conditions, for carrying out body paintings. This is because the fruit provides, by pressing, a colorless liquid juice which is rich in glycosides of the iridoid group (and other compounds which are members of the chemical group of the monoterpenoids). The combination of this juice is spontaneously modified, the geniposide being converted to genipin by hydrolysis, which turns dark blue in color by reaction, in the presence of oxygen, with the amino acids or the proteins present in the medium, namely this juice. The coloring of the genipin blue by the abovementioned reaction can also take place at the surface of the support to which the juice is applied immediately after it is obtained.

Consequently, commercial solvent extracts of Genipa americana, such as those sold by SCRD (Le Havre, France), are naturally blue in color.

It is thus understood that, for cosmetic use, it does not appear desirable to use the colorless juice or extract obtained from the fruit of the plant as such as, on contact with the skin, genipin becomes attached in a semipermanent fashion, like a temporary tattoo. The skin stains thus formed can endure and remain visible for several days and up to two to three weeks.

Consequently, in order to use the extract of this plant as coloring material, in particular in products intended to be applied to the skin or superficial body growths, it is essential beforehand, in order to avoid this undesirable effect, to use an extract in which the genipin has already reacted as indicated above to develop the color. Such extracts are available commercially, as indicated above. In addition, for use in a coloring composition, such as a makeup product, it is subsequently necessary to render this plant dye insoluble, as explained above.

It is precisely the problem of rendering insoluble genipin or a plant extract comprising genipin, geniposidic acid or geniposide which the present invention intends to solve, with the aim of providing a stable coloring material which can be used in different makeup products and which, unlike the products conventionally used in tattooing, must not stain the skin or become semipermanently attached thereto, in order to be able to be easily removed during makeup removal.

This rendering insoluble has been able to be carried out by the Applicant and the coloring materials which have been developed form the subject matter of the present invention.

Various Amerindian traditions report techniques for the adsorption on clay of plant compounds. Mention may be made, for example, of “Maya Blue”, which has formed the subject of research of an archeological nature. The preparation of this coloring material consists of an adsorption by the dry route of natural indigo on a clay of palygorskite type.

Other plant sources have also been able to give clays colored green, yellow or red but, to date, while the analysis of the archeological record clearly reports such products, the precise methods and ingredients for obtaining these compounds have not been clearly elucidated.

Thus, the present invention relates, as novel product, to a coloring material resulting from rendering insoluble a dye derived from geniposide or genipin or geniposidic acid or a coloring plant extract including at least geniposide and/or genipin and/or geniposidic acid.

It also relates to a process for preparing such a coloring material.

It also relates to the use of this coloring material, in particular in the cosmetics field.

More specifically, the invention relates to a coloring material comprising a colored product bound to a solid substrate, wherein said colored product is a colored derivative of geniposide and/or genipin and/or geniposidic acid and/or a coloring extract of a plant comprising at least one of the abovementioned products and wherein said solid substrate is based on silicate and includes at least one metal cation.

The invention also relates to the process for the preparation of this coloring material, said process comprising:
a stage of rendering insoluble a colored product derived from geniposide and/or genipin and/or geniposidic acid and/or an extract of a plant comprising one of the abovementioned products, starting from an aqueous solution of said colored product, by formation of an aqueous suspension of colored solid particles, said colored solid particles being composed of a solid substrate based on silicate including at least one metal cation to which said colored product is bound, and
a stage of recovery of the colored solid particles obtained during the stage of rendering insoluble.

The invention relates in addition to the use of the coloring material in the preparation of a coloring composition, in particular of a cosmetic composition.

The invention also relates to a method for making up the skin or superficial body growths which comprises the topical application of a cosmetic composition comprising said coloring material or obtained according to said preparation process.

Other characteristics and advantages of the invention will become apparent in the detailed description which follows.

As set out above, the invention results from the discovery by its inventors that it is possible to render insoluble the colored products derived from geniposide and/or genipin and/or genoposidic acid and/or an extract of a plant comprising them by fixing them to a support based on silicate including at least one metal cation, so as to prepare a coloring material in the solid form, one of the advantages of which is that of not becoming attached in a lasting fashion to the skin when it is used as coloring material in a cosmetic composition.

Such coloring materials constitute novel products.

The colored product can be any colored derivative of geniposide or of genipin or geniposidic acid.

As set out above, such colored derivatives are known in the prior art. It is the same for the extracts of plants comprising geniposide or genipin or geniposidic acid and for the colored materials obtained from these extracts.

The invention thus lies essentially in the preparation of a product resulting from the fixing of these various colored materials, obtained by reaction from geniposide and/or genipin and/or geniposidic acid, to the very specific solid substrate which makes it possible to render insoluble the colored product, preventing it from becoming attached in a lasting fashion to the skin, which makes it possible in particular to envisage applications in the makeup field.

The cations included in the silicate belong to a wide range. However, advantageously, these cations belong to the group consisting of Al³⁺, Fe³⁺, Mg²⁺, Fe²⁺ and Li⁺. Preferably, a cation belongs at least to the group consisting of Mg²⁺ and Al³⁺.

The choice will advantageously be made, as solid substrates based on silicate including at least one metal cation, of a clay.

This clay can be a natural clay or an artificial clay.

It is well known that clays are hydrated phyllosilicates formed by an arrangement of tetrahedra in hexagonal networks to form two-dimensional sheets, which themselves are combined with octahedral sheets formed of a central cation among those mentioned above and of six OH⁻ ions.

These sheets constitute the first structural level of the clays. These sheets gather into particles, which themselves gather into aggregates.

The clays, whether natural or synthetic, can be chosen within a very wide range. However, according to a particularly advantageous alternative form of the invention, the clay is chosen from “expandable” or “swelling” clays.

According to another advantageous alternative form of the invention, the clay is chosen from those which exhibit a high mean ion-exchange capacity, preferably a capacity of greater than 50 meq. per 100 g of the clay forming the solid substrate.

An example of such clays is composed of those belonging to the family of the smectites and those belonging to the family of the vermiculites.

Advantageously, the clay is chosen from those belonging to the family of the smectites and more particularly from saponites, hectorites, montmorillonites and bentonites.

In a particularly advantageous way, laponite is chosen as clay.

According to another advantageous alternative form, the solid substrate is obtained by reaction of magnesium sulfate with sodium silicate.

According to a particularly advantageously alternative form of the invention, the coloring material is in the form of a pulverulent solid advantageously exhibiting a mean particle size, defined by its median diameter (D₅₀), of less than 30 μm, preferably of between 0.01 μm and 10 μm.

The median diameter (D₅₀) of a powder sample is defined as the diameter below which 50% of the weight of the sample tested is found.

As set out above, geniposide, genipin and geniposidic acid are not naturally colored but result in colored derivatives by processes which have already been described in the literature.

The invention is targeted, as colored products, at all the colored derivatives of geniposide or genipin or geniposidic acid.

The colored product used is advantageously obtained from a plant extract comprising geniposide, genipin or geniposidic acid, in particular from a plant belonging to the botanical family of the Rubiaceae.

According to an alternative form of the invention, the colored product used is obtained by reaction, in the presence of oxygen, of genipin with a primary amine group carried, for example, by an amino acid and/or a protein.

Advantageously, the colored product is a colored plant extract, the coloring of which is obtained spontaneously, under the effect of oxygen and/or ambient air, by reaction of the genipin present in the extract with amino acids and/or proteins carrying a primary amine group present in the same extract.

According to another alternative form of the invention, the colored product is obtained by reaction, in an acidic medium and in the absence of oxygen, of geniposidic acid, which can be present in the plant extract or obtained from the geniposide present in the plant extract, with at least one primary amine group carried, for example, by an amino acid, such as arginine or glutamic acid, as set out above.

Advantageously, the choice will be made to use a colored product obtained from a plant extract comprising geniposide and/or genipin and/or geniposidic acid, in particular of a plant chosen from the botanical family of the Rubiaceae.

As set out above, a number of plants of this family which can result in colored products are known, such colored products having already been used in the prior art, in particular for producing dyes and in particular body dyes.

According to an advantageous alternative form, the plant belongs to the genus Genipa, Gardenia, Rothmannia, Adenorandia or Cremaspora.

Mention is made, among the preferred plants for producing the coloring materials of the invention, of Genipa americana and Gardenia jasminoides and very particularly Genipa americana.

For both these plants, the extract is advantageously an extract of the whole fruit or of at least a part of the fruit.

The choice is preferably made of an extract of the pulp and/or of the seeds of the fruit.

According to a particularly advantageous alternative form, the extract is a juice of the fruit of one of these plants, obtained in particular by pressing, or an aqueous extract, obtained in particular by steeping or macerating, the whole fruit or a part of the fruit.

In both cases and very particularly in the case of Genipa americana, the extract is preferably obtained from the fruit of the plant and more particularly by extraction of the whole fruit or also of the pulp and/or of the seeds of the fruit.

Extraction is carried out by pressing the fruit or at least a part of the fruit or also by macerating or steeping in a polar solvent, preferably an aqueous medium.

The extract obtained can be used directly or else can be treated so as to hydrolyze the geniposide to genipin or geniposidic acid.

The extract can thus be treated enzymatically using a β-glucosidase, to hydrolyze the geniposide to give genipin, or chemically, for example according to the abovementioned publication Moritome et al., to hydrolyze the ester functional group of geniposide to give the acid functional group of geniposidic acid.

The extract can also be brought into the presence of oxygen, in particular in ambient air, so as to allow the extract to become colored by reaction of the genipin with amino acids having a primary amine group or the proteins comprising such a group which are present in the extract.

It is also possible to enrich the plant extract using compounds carrying a primary amine group, in particular amino acids carrying such groups, so as to obtain a particular hue.

As explained above, the color of the product depends on the reactants and on the conditions of the reaction resulting in said colored product.

According to a first alternative form, starting from genipin, optionally obtained from geniposide and/or a plant extract comprising it, the colored product obtained under the conditions described above is blue in color. The hue of the blue thus obtained can vary from sky blue to blue-black and depends in particular on the composition with regard to amino acids or with regard to proteins which have reacted with the genipin.

According to a second alternative form, starting from geniposidic acid, optionally obtained from geniposide and/or a plant extract comprising it, the colored product obtained under the conditions described above is red in color. The hue of the red thus obtained also varies according to the amino acid which has reacted with the geniposidic acid. This red hue can be shaded by adding, to the reaction medium, a sugar of pentose type, such as D-ribose or D-xylose, as is described in the text of the Japanese patent 2873518.

The colored product, whether obtained by one or other of the alternative forms, is soluble in water.

The colored product is used for the preparation of the coloring materials which are a subject matter of the invention.

As set out above, the invention also relates to a process for the preparation of the coloring materials defined above.

According to this process, a suspension of colored solid particles based on silicate comprising at least one metal cation, to which particles the colored product defined above is fixed, is prepared.

This suspension is produced from an aqueous solution of a colored product obtained from geniposide and/or genipin and/or geniposidic acid and/or a plant extract comprising it/them.

The colored product fixed to said particles is thus rendered insoluble.

The colored solid particles are subsequently recovered by removing the aqueous medium by any appropriate solid/liquid separating means, in particular by settling and/or filtration.

As emerges from the account which follows, this suspension of colored particles in an aqueous medium can be obtained either from preexisting solid particles, in particular clay particles, to which the colored product becomes fixed, or by forming in situ the colored solid particles based on silicate within the aqueous solution of colored product, in particular by reaction of a salt of a metal cation with an aqueous solution of a silicate salt, for example a sodium silicate.

In the case where a preformed support is used, the aqueous suspension is prepared, preferably with stirring, by bringing the aqueous solution of the colored product into contact with the solid substrate as defined above, in particular with the clay.

According to this process, the solid substrate is added to the aqueous solution in the form of a powder or of an aqueous suspension.

According to a preferred implementation, an amount of solid substrate of between 50% and 100% of the dry weight of the colored product as defined above is added to said aqueous solution.

According to an advantageous alternative form of the above process, an additional stage of treatment in an acidic medium, preferably after the stage of bringing the aqueous solution into contact with the solid substrate, is carried out.

In the case where the colored solid particles based on silicate are prepared in situ, a process comprising the following stages is advantageously carried out:
addition of a metal cation salt, preferably a magnesium or aluminum sulfate, to an aqueous solution of the colored product as defined above,
bringing the aqueous solution thus obtained into contact, with stirring, with an aqueous solution of a silicate, preferably an alkali metal silicate, such as sodium silicate, said mixture reacting during said contacting operation to precipitate said colored solid particles in situ.

In each of the alternative forms of the process set out above, the aqueous solution of the colored product is advantageously obtained by dissolving, in water, a plant extract as defined above and in particular a Genipa americana extract.

The process in addition advantageously comprises at least one stage of settling and/or washing and/or filtering and/or drying and/or spraying or atomizing intended to collect a powder formed of colored material essentially composed of the solid substrate to which the colored product is fixed.

In each of the two alternative forms of the process, the broad outlines of which are marked out above, a person skilled in the art can vary the various operating conditions and parameters.

The latter will easily understand that heating proves to be advantageous both for improving the distribution of the colored product in the aqueous medium and for improving the fixing to the solid substrate. However, a substantially equivalent result can be obtained without heating as regards the fixing but with slower kinetics.

Furthermore, it has been possible to observe an improvement in the fixing by addition of acid, in particular tartaric acid, to the aqueous medium. This addition is advantageously carried out after the aqueous medium comprising the colored material has been brought into contact with the solid substrate. However, it is apparent that the acid can also be introduced without disadvantage before the contacting operation. Furthermore, the acid can be replaced by a salt of this acid. Tests carried out by the inventors have furthermore shown that recourse to an acid or an acid salt is favorable to the obtention of bigger particles.

In addition, tests carried out by the inventors have shown that, in the case of an extract of commercial origin comprising in particular glucosides, it is advantageous to carry out a reaction for the hydrolysis of the glycosides, which improves the fixing of the colored product to the solid substrate.

The same types of effects of the various operating parameters were observed for both alternative forms of the process.

Another subject matter of the invention relates to the use of the coloring material of the invention in coloring compositions capable of including it.

The coloring materials of the invention are of particular use in cosmetic compositions and in particular in cosmetic compositions intended for making up the skin or superficial body growths.

Thus, the invention relates to coloring compositions, in particular cosmetic compositions for making up the skin or superficial body growths, comprising the coloring material as defined above.

Within the meaning of the invention, the coloring compositions are those in which the coloring agent retains its coloring properties without the other compounds denaturing the specific structure thereof.

In the cosmetics field, the coloring composition will generally be a product for making up the skin or superficial body growths, in particular a mascara, a foundation, an eye shadow, an eyeliner, a nail varnish or a loose or compact powder.

The cosmetic compositions comprising the coloring material of the invention can comprise a cosmetically acceptable active agent and at least one cosmetically acceptable excipient chosen from pearlescent agents, polymers, surface-active agents, rheology agents, fragrances, electrolytes, pH adjusters, antioxidants, preservatives and their mixtures, and optionally other coloring agents.

The invention also relates, in a particularly preferred way, to the use of the coloring material of the invention, prepared from extracts of Rubiaceae comprising geniposide and/or genipin and/or geniposidic acid, in particular extracts of Genipa americana or Gardenia jasminoides, as coloring agent, in coloring compositions capable of including it and more particularly in cosmetic compositions, in particular compositions intended for making up the skin or superficial body growths, such as the eyelashes.

A person skilled in the art will easily understand that the amount of coloring material present in the compositions of the invention is very largely dependent on the type of composition and on the effect desired.

The invention also relates to a method for making up the skin or superficial body growths, in particular the eyelashes, hair or nails, comprising the application, to at least a part of the skin or superficial body growths, of a composition as has been described above.

Finally, as set out above, the colored materials of the invention also have applications in fields other than that of cosmetics, in particular in that of foodstuffs or decorative paint.

### EXAMPLES

### Example 1

### Fixing of a Genipa americana Extract to Laponite

100 g of a commercial extract of Genipa americana fruit in the form of an atomized powder, obtained from SCRD (Le Havre, France), are dissolved in 10 liters of osmotically treated water brought to 60° C. and then, when dissolution is complete, an amount of laponite of between 50% and 100% of the weight of the dry extract weighed at the start, i.e. 50 g, is added, still with stirring (the proportions can vary according to the richness of the starting extract and it will be up to a person skilled in the art to adjust these proportions according to the quality of the materials available to him). The liquid is subsequently brought to a temperature of 80° C. approximately. When the clay particles begin to become charged with the colored plant compounds, which is observable through the fact that the liquid becomes much clearer, an amount of tartaric acid of, in the present case, between 10 and 20 g is added, the effect of which is to improve the phenomenon of adsorption of the plant compounds on the clay substrate. Separation by settling is allowed to take place. The clay/organic materials complex is deposited, while the supernatant liquid is a dirty yellow.

The deposit is subsequently washed by addition of clean water, two to three times, until the aqueous wash liquors, after separation by settling, no longer exhibit any acidic nature and are no longer colored. The deposit is collected on a filter, dried and then reduced to a powder. Finally, the powder is again washed using an aqueous/alcoholic solution, in order to remove therefrom possible organic traces which were not fixed to the clayey support.

### Example 2

### Fixing of a Genipa americana Extract to a Solid Substrate Formed In Situ

100 g of atomized powder of the same atomized commercial extract of Genipa americana fruit as above are dissolved in 10 liters of osmotically treated water brought to approximately 40° C., then, when dissolution is complete, 100 g of commercial magnesium sulfate assaying 16% of MgO are added and, finally, an aqueous sodium silicate solution assaying 38-40% by weight of silicate is added. The combined mixture is subsequently heated up to 80° C. until significant flocculation appears. The liquid is allowed to separate by settling. The precipitate is washed by successive additions of clean water, followed by separations by settling. The precipitate is subsequently collected on a filter and dried. It can subsequently be washed with alcohol as above.

### Example 3

### Makeup Composition Comprising the Coloring Material According to the Invention

The coloring material obtained according to example 2 is a powder which is blue-black in color. It is added to a mascara, the formula of which is described below:

| | |
|---|---|
| Coloring material according to the invention | 8 |
| Excipients for mascara (including fragrances and | q.s. |
| preservatives) | |
| Water | q.s. 100 |

The mascara thus obtained is blue-black in color.

What is claimed:

1. A coloring material comprising a colored product bound to a solid substrate, wherein said colored product is a colored derivative of a product or a mixture of products selected from the group consisting of geniposide, genipin, geniposidic acid and an extract of a plant comprising at least one of the abovementioned products, and wherein said solid substrate is based on silicate and includes at least one metal cation.

2. The coloring material according to claim 1, wherein said solid substrate comprises at least one cation selected from the group consisting of Al³⁺, Fe³⁺, Mg²⁺, Fe²⁺ and Li⁺.

3. The coloring material according to claim 1, wherein said solid substrate is a clay.

4. The coloring material according to claim 3, wherein said clay is a smectite.

5. The coloring material according to claim 3, wherein said clay is laponite.

6. The coloring material according to claim 1, wherein said solid substrate is obtained by reaction of magnesium sulfate with sodium silicate.

7. The coloring material according to claim 1, wherein said solid substrate is in the form of a powder exhibiting a mean particle size (D₅₀) of less than 30 μm.

8. The coloring material according to claim 1, said plant being chosen from the botanical family of the Rubiaceae.

9. The coloring material according to claim 8, said plant belonging to the genus Genipa, Gardenia, Rothmannia, Adenorandia or Cremaspora.

10. The coloring material according to claim 1, said colored product being obtained by reaction, in the presence of oxygen, of genipin with at least one primary amine group.

11. The coloring material according to claim 1, said colored product being obtained by reaction, in an acidic medium and in the absence of oxygen, of geniposidic acid with at least one primary amine group.

12. The coloring material according to claim 10, said primary amine group being carried by an amino acid, a protein or both.

13. The coloring material according to claim 12, said amino acid or said protein being present in an extract of a plant comprising a product or a mixture of products selected from the group consisting of geniposide, genipin and geniposidic acid.

14. The coloring material according to claim 8, said extract being a Genipa americana extract.

15. The coloring material according to claim 8, said extract being a Gardenia jasminoides extract.

16. The coloring material according to claim 14, said extract being an extract of the whole fruit of said plant or at least a part of said fruit.

17. The coloring material according to claim 14, said extract being an extract of the pulp, of the seeds of the fruit or both.

18. The coloring material according to claim 14, said extract being the juice of the fruit, obtained by pressing, or an aqueous extract, obtained by steeping or macerating the fruit or a part of the fruit.

19. A method for preparing a coloring material of claim 1 comprising:
rendering insoluble a colored product as defined in claim 1, starting from an aqueous solution of said colored product, by formation of an aqueous suspension of colored solid particles, said colored solid particles being composed of a solid substrate based on silicate including at least one metal cation to which said colored product is bound, and
recovering the colored solid particles obtained by rendering the colored product insoluble.

20. The process according to claim 19, wherein the aqueous suspension is prepared, with stirring, by bringing said aqueous solution of said colored product into contact with a solid substrate as defined in claim 1.

21. The process according to claim 20, wherein said solid substrate is introduced into said aqueous solution in the form of a powder or of an aqueous suspension of said solid substrate, the amount of solid substrate being between 50% and 100% of the dry weight of the colored product.

22. The process according to claims 19, which additionally comprises a stage of treatment in an acidic medium carried out after the stage in which said aqueous solution is brought into contact with said solid substrate.

23. The process according to claim 20, wherein the aqueous suspension is formed by a process comprising:
adding a salt of said metal cation to said aqueous solution of the colored product as defined in claim 1,
bringing the aqueous solution thus obtained into contact, with stirring, with an aqueous solution of a silicate salt, said mixture reacting during said contacting operation to precipitate said colored solid particles in situ.

24. The process according to claim 19, wherein said aqueous solution of the colored product is obtained by dissolving, in water, an extract as defined in claim 1.

25. The process according to claim 19, further comprising at least one step selected from the group consisting of washing, filtering, drying, spraying and atomizing intended to collect a powder essentially composed of said solid substrate to which said colored product is fixed.

26. A composition comprising the coloring material as defined in claim 1.

27. The composition according to claim 26, wherein it is a cosmetic composition selected from the group consisting of a mascara, a foundation, an eye shadow, an eyeliner, a nail varnish, a loose powder and a compact powder.

28. The composition according to claim 26, which is selected from a paint and a decorative paint.

29. A method of cosmetic care for making up the skin or superficial body growths, which comprises the topical application of a cosmetic composition comprising the coloring material as defined in claim 1.
